(19) United States
(12) Patent Application Publication
(10) Pub. No.: US 2026/0114788 A1
(43) Pub. Date: Apr. 30, 2026

(54) DEVICE FOR PLACEMENT OF AN INTRANASAL ELECTRODE

(71) Applicant: Northwestern University, Evanston, IL (US)

(72) Inventors: Bruce Kuang-Huay TAN, Evanston, IL (US); Philippe GENEREUX, Evanston, IL (US); Josh MILLER, Evanston, IL (US); Michael GARRETT, Evanston, IL (US); James HENDERSON, Evanston, IL (US); Christina Maria ZELANO, Evanston, IL (US); Gregory LANE, Evanston, IL (US)

(73) Assignee: Northwestern University, Evanston, IL (US)

(21) Appl. No.: 19/368,664

(22) Filed: Oct. 24, 2025

Related U.S. Application Data
(60) Provisional application No. 63/712,064, filed on Oct. 25, 2024.
(60) Provisional application No. 63/785,870, filed on Apr. 9, 2025.

(51) Int. Cl. A61B 5/00; A61B 1/00; A61B 1/005; A61B 1/233

(52) U.S. Cl. CPC A61B 5/4011; A61B 1/00128; A61B 1/0014; A61B 1/005; A61B 1/233; A61B 5/6847; A61B 5/6867; A61B 2560/0468; A61B 2562/043

(57) ABSTRACT

Provided herein is a nasal applicator device for precision placement of a nasal cavity device in an olfactory cleft of a subject comprising: a handle extending between a handle distal end and a distal proximal end; one or more controllers operably connected to the handle; an applicator arm connected to the handle distal end, a distal end effector configured to operably connect to the applicator arm to guide placement of the nasal cavity device; a stabilizer operably connected to an applicator arm distal end, wherein during use the stabilizer is configured to stably immobilize at least a portion of the nasal cavity device within the olfactory cleft of the subject.

### CROSS-REFERENCE TO RELATED APPLICATIONS

This application claims the benefit of priority to U.S. Provisional Ser. No. 63/712,064 , filed Oct. 25, 2024 and U.S. Provisional Ser. No. 63/785,870 , filed Apr. 9, 2025, each of which is hereby incorporated by reference in its entirety.

### BACKGROUND OF INVENTION

The study and treatment of human olfaction and its pathologies has been hampered by the difficulty of direct imaging or recording of the primary olfactory peripheral and central structures: the olfactory epithelium and the olfactory bulb. These structures are located at the top of the nasal cavities, with the olfactory epithelium concentrated in the dorsal aspect of the olfactory cleft, and the olfactory bulb approximately 1-2 mm above it, with the two separated by a thin perforated section of the skull called the cribriform plate, through which the olfactory nerves connect the two structures. These structures are the first areas of odor processing and understanding them is important to understanding the human sense of smell. By placing a nasal cavity device, such as, for example, an electrode—a very thin wire with electrical contacts along its tip—up into the olfactory cleft, one can record signals from both the epithelium and the bulb. These signals are produced during the sensing and processing of odors, and they provide a wealth of data that can be used to understand the system in health and disease, allowing for diagnostics and interventions in olfactory disease. However, placement of a nasal cavity device at this location is technically challenging. This area of the nose is rarely accessed, even by ENTs, except during skull-base surgery when it is typically destroyed to allow access to the brain (to remove tumors, for example) and instrumentation to access this area in an awake patient is lacking. The nasal cavity device, for example, an electrode, must be precisely placed in a very small area that is unfamiliar to common ENT practice. In order to place a nasal cavity device, the ENT must maneuver a nasal endoscope in one hand and carefully navigate a tortuous nasal cavity with the nasal cavity device held by a clamp in the other hand, then place and release the nasal cavity device, and remove the clamp without moving the nasal cavity device from its desired placement. The nasal cavity device can be floppy and otherwise difficult to handle, resulting in inexact placement, or can require adjustment. Parts of the nasal cavity are very sensitive, so adjustments can be painful. The nasal applicator device, disclosed herein, solves these problems by allowing one-handed control over placement, bending a nasal cavity device so it conforms to the direction of the olfactory cleft and stabilizes the nasal cavity device at the desired placement without obstructing the nasal passage.

Provided herein are devices and related methods that solves the lack of suitable instrumentation for accessing and placing nasal cavity devices into the human olfactory cleft. The nasal applicator devices and methods facilitate important and fundamental understanding of human olfactory disorders and treatment of human olfactory disorders using, for example, direct electrical stimulation.

### SUMMARY OF THE INVENTION

Aspects disclosed herein include nasal applicator devices that allow and perform one-handed placement, adjustment, and stabilization of a nasal cavity device, for example, an intranasal electrode, or similar device such as a catheter, such that the far (or distal) end of the nasal cavity device is placed at the sphenoid sinus wall, or at another location in the olfactory cleft, such that the active (recording or stimulating) aspect of the nasal cavity device is placed along the nasal cleft below the cribriform plate, or at another location in the olfactory cleft, and the stabilizing assembly is placed at the opening of the olfactory cleft, for the purposes of recording electrophysiological signals, delivering electrical stimulation, delivering drugs, recording impedance or voltammetry or patch-clamp measurements, in humans.

The device (or applicator), also referred to herein as a “a nasal applicator device”, comprises a handle extending between a handle distal end and a distal proximal end; one or more controllers operably connected to the handle; an applicator arm connected to the handle distal end, a distal end effector configured to operably connect to the applicator arm to guide placement of the nasal cavity device; a stabilizer operably connected to an applicator arm distal end, wherein during use the stabilizer is configured to stably immobilize at least a portion of the nasal cavity device within the olfactory cleft of the subject, and is designed for use in precision placement of a nasal cavity device. The applicator is designed such that the user places the nasal cavity device into the applicator in preparation for placement, then performs the placement with the applicator in one hand and, optionally, a nasal endoscope in the other. Using the endoscope to provide a visual guide, the user guides the applicator with loaded nasal cavity device up into the nasal cavity, so that the tip of the applicator is at the anterior opening of the olfactory cleft. Then, using one hand, the user can advance the nasal cavity device through the nasal applicator device so that the nasal cavity device, optionally, an electrode, moves up and into the olfactory cleft until the tip of the nasal cavity device touches the sphenoid sinus wall and the nasal cavity device, optionally, electrode contacts, are along the ventral aspect of the cribriform plate, at the olfactory epithelium. The user can adjust the position of the nasal cavity device using a thumb screw on the handle of the nasal applicator device to advance or retract the nasal cavity device through the applicator. Once the nasal cavity device is in place, the user engages, including by a push force, a finger slide controller on the handle of the nasal applicator device to deploy the stabilizer assembly at the opening of the olfactory cleft. The stabilizer assembly attaches to the nasal cavity device, optionally an electrode wire, and comprises, for example, a sponge that inflates with mucosal fluids until it pushes against the nasal cavity walls with sufficient force to hold the nasal cavity device in place for the duration of the procedure. The finger-slide action of deploying the stabilizer assembly simultaneously detaches the nasal cavity device and the stabilizer assembly from the nasal applicator device such that the nasal applicator device can be removed from the nasal cavity and the nasal cavity device stays in place. A version of the nasal applicator device does not use a finger slide to deploy the stabilizer assembly, and instead uses a physical stopper attached to the nasal cavity device, which mechanically deploys the stabilizer assembly when the nasal cavity device is extended to the preferred placement.

The nasal applicator device and methods disclosed herein, facilitate placement of a nasal cavity device that can perform or facilitate, for example, the following in humans:
record electrophysiological signals from the olfactory bulb;
record electrophysiological signals from the olfactory epithelium;
stimulate the olfactory bulb;
stimulate the olfactory epithelium;
deliver drugs to the olfactory epithelium through the catheter;
provide objective electrical measurement of olfactory ability;
mapping of the location of the olfactory epithelium and individual olfactory receptors;
diagnosis of olfactory disorders, including differentiating various causes of olfactory disorders;
treatment of olfactory disorders through electrical stimulation of the olfactory epithelium and bulb;
treatment of neurologic disorders that are directly connected to the olfactory epithelium and bulb;
can determine whether olfactory dysfunction is of central or peripheral origin;
packages electrode directional guidance, placement and stabilization into a single device;
allows one-handed control for rapid, precise placement and stabilization of the electrode against the cribriform plate; and/or
eliminates the need for customized fastening of stabilizers to electrode.

This nasal applicator device is especially important for reliable and efficient nasal placement of recording or stimulating nasal cavity devices, such as, for example, electrodes, into the human olfactory system. In the desired placement location, both signals of the peripheral and central olfactory system can be captured. The ability to reliably and comfortably place a nasal cavity device, for example, an electrode in this location facilitates fundamental and clinical understanding of human olfactory signal processing.

In an embodiment, disclosed herein is a nasal applicator device for precision placement of a nasal cavity device in an olfactory cleft of a subject comprising: a handle extending between a handle distal end and a distal proximal end; one or more controllers operably connected to the handle; an applicator arm connected to the handle distal end; a distal end effector configured to operably connect to the applicator arm to guide placement of the nasal cavity device; and a stabilizer operably connected to an applicator arm distal end, wherein during use the stabilizer is configured to stably immobilize at least a portion of the nasal cavity device within the olfactory cleft of the subject.

In an embodiment, optionally, the nasal applicator device further comprises a hollow tip positioned at the applicator arm distal end configured to stably immobilize the nasal cavity device in the applicator arm.

In an embodiment, optionally, the distal end effector comprises a tip guide. The tip guide may be detachable from the applicator arm. Optionally, in an embodiment, the distal end effector comprises a flex clip. The flex clip may be configured to retract upon mechanical actuation of the one or more controllers wherein retraction causes the flex clip to secure to the tip guide and the nasal cavity device.

In an embodiment, optionally, the applicator arm comprises an applicator channel cover that is releasably connected to the nasal cavity device. In embodiments, the one or more controllers is configured to rotate the applicator channel cover in order to release the nasal cavity device from the nasal applicator device after the nasal cavity device has been precisely placed in the olfactory cleft of a subject.

In an embodiment, optionally, the stabilizer is part of a top stabilizer assembly. The stabilizer may be controllably positionable in the nasal cavity at a user-selected location according to an anatomical parameter and the stabilizer may have size configured to extend between the nasal cavity device outer surface and an inner surface of the nasal cavity. The stabilizer provides sufficient contact between the nasal cavity device and the inner walls of the nasal cavity so as to reliably position the electrophysiological device, also referred to herein as the nasal cavity device. The stabilizer may be controllably positioned by any number of means, including by a tensile force exerted between the stabilizer and the underlying nasal cavity device, such that the stabilizer can translate over the surface, but once positioned in the nasal cavity, will not move. Similarly, a fastener such as an adhesive may be used to ensure the stabilizer does not move during use, or similarly, a fastener such as a rivet or screw secured through the wall of the sinus cavity may be used to ensure the stabilizer does not move during use, particularly in cases of long-term placement and use. For example, for long term placement applications on the order of years, including for wireless devices where it is desired to apply stimulation over years, such a fastener through the sinus cavity of the wall assists in long term and reliable device positioning.

In embodiments, the top stabilizer assembly comprises various components. For example, the top stabilizer assembly may comprise the flex clip, the tip guide and the stabilizer. Optionally, in an embodiment, the flex clip, the tip guide, and the stabilizer are operably connected.

The stabilizer may further be configured to apply tension upon deployment including spring-based tension or a mechanical material-based tension.

The stabilizer may comprise a material selected from the group of: a silicone, a urethane, an elastomer, a polymer, or any combination of these. The stabilizer may comprise an absorptive sponge, an inflatable catheter, a flap, a ring, a wedge, a balloon, or a u-shape stabilizer. Any geometry or shape of the stabilizer is envisioned herein, so long as the geometry is capable of applying tension between the flexible tube of the nasal cavity device and an inner surface of a subject's nasal cavity upon deployment.

The stabilizer may comprise an absorptive material comprising dehydrated hydroxylated polyvinyl acetate, synthetic biodegradable fragmenting foam, oxidized nitrocellulose, cotton, non-woven gauze, polyether-polyurethane, medical-grade foam, silicone, polyvinyl acetate, a resorbable oxidized cellulose, a hemostatic matrix, or a thrombin soaked hemostatic device prepared purified porcine skin gelatin. The absorptive material may absorb, for example, nasal fluids or other biological fluid present in the subject. Of course, the devices are also compatible with an externally-applied liquid, such as saline.

The stabilizer may have a thickness greater than or equal to 0.5 mm and less than or equal to 2.2 mm. The thickness of the stabilizer can be selected based on an individual subject's nasal anatomy.

In an embodiment, the nasal applicator device may further comprise an applicator channel longitudinally aligned with the applicator arm, wherein the applicator channel longitudinally extends from the handle distal end and is configured to connect to a nasal endoscope to visualize the nasal cavity of the subject. In embodiments, therefore, the nasal applicator device allows for one handed placement of a nasal cavity device of embodiments disclosed herein.

In an embodiment, optionally, the applicator arm or the tip guide has a curved geometry or malleability to accommodate a mechanical rotation of the nasal cavity device at an external angle of between 45° and 90° for controllable placement of at least a portion of the nasal cavity device within the olfactory cleft of the subject.

In an embodiment, optionally, the one or more controllers are configured to advance the nasal cavity device within the nasal cavity.

In an embodiment, optionally, the one or more controllers are configured to retract the nasal cavity device within the nasal cavity.

In an embodiment, optionally, the one or more controllers are configured to engage the stabilizer within the nasal cavity. Thus, the one or more controllers may be configured to expand the stabilizer within the nasal cavity in order to stably immobilize the nasal cavity device within the nasal cavity. Optionally, the one or more controllers may be configured to retract or collapse the stabilizer, for example, to its original size, during removal or repositioning of the nasal cavity device.

In an embodiment, optionally, the one or more controllers are configured to release the top stabilizer assembly.

In an embodiment, optionally, the one or more controllers are configured to retract the flex clip.

In an embodiment, optionally, the one or more controllers are configured to secure the flex clip to the tip guide.

In an embodiment, optionally, the one or more controllers are configured to rotate the applicator channel cover.

In an embodiment, the one or more controllers are configured to advance the nasal cavity device between 10 mm to 40 mm so as to place at least a portion of the nasal cavity device within the olfactory cleft of the subject. Optionally, the one or more controllers are configured to advance the nasal cavity device between 15 mm to 40 mm so as to place at least a portion of the nasal cavity device within the olfactory cleft of the subject. Optionally, the one or more controllers are configured to advance the nasal cavity device between 20 mm to 40 mm so as to place at least a portion of the nasal cavity device within the olfactory cleft of the subject. Optionally, the one or more controllers are configured to advance the nasal cavity device between 25 mm to 40 mm so as to place at least a portion of the nasal cavity device within the olfactory cleft of the subject. Optionally, the one or more controllers are configured to advance the nasal cavity device between 30 mm to 40 mm so as to place at least a portion of the nasal cavity device within the olfactory cleft of the subject. Optionally, the one or more controllers are configured to advance the nasal cavity device between 35 mm to 40 mm so as to place at least a portion of the nasal cavity device within the olfactory cleft of the subject. Optionally, the one or more controllers are configured to advance the nasal cavity device between 10 mm to 35 mm so as to place at least a portion of the nasal cavity device within the olfactory cleft of the subject. Optionally, the one or more controllers are configured to advance the nasal cavity device between 10 mm to 30 mm so as to place at least a portion of the nasal cavity device within the olfactory cleft of the subject. Optionally, the one or more controllers are configured to advance the nasal cavity device between 10 mm to 25 mm so as to place at least a portion of the nasal cavity device within the olfactory cleft of the subject. Optionally, the one or more controllers are configured to advance the nasal cavity device between 10 mm to 20 mm so as to place at least a portion of the nasal cavity within the olfactory cleft of the subject. Optionally, the one or more controllers are configured to advance the nasal cavity device between 10 mm to 15 mm so as to place at least a portion of the nasal cavity device within the olfactory cleft of the subject. Preferably, the one or more controllers are configured to provide a reliable and maintainable position of an intranasal electrode within the olfactory cleft, including for electrical interfacing between the electrode and biological components of the olfactory cleft, including for when the electrode is detached from other portions of the device, including the handle and controller(s).

In an embodiment, the one or more controllers comprises a thumb screw for nasal cavity device detachment or advancement.

In an embodiment, the one or more controllers comprises a finger slide for nasal cavity device detachment or advancement.

In an embodiment, the one or more controllers comprises a deployment trigger for nasal cavity device detachment or advancement.

In embodiment, the one or more controllers comprises an advancement wheel for nasal cavity device detachment or advancement.

In an embodiment, optionally, the one or more controllers is selected from the group consisting of: a thumb screw, a finder slide, an advancement wheel and a deployment trigger for nasal cavity device detachment or advancement.

In an embodiment, the finger slide control or the deployment trigger is configured to detach the nasal cavity device from the nasal applicator device, such that the nasal applicator device may be removed from the nasal cavity and the nasal cavity device remains in the nasal cavity of the subject.

In an embodiment, the finger slide control or the deployment trigger is configured to detach the top stabilizer assembly from the nasal applicator device, such that the nasal applicator device can be removed from the nasal cavity and the top stabilizer assembly remains in the nasal cavity of the subject.

In an embodiment, the finger slide control or the deployment trigger is configured to detach the stabilizer from the nasal applicator device such that the nasal applicator device can be removed from the nasal cavity and the stabilizer remains in the nasal cavity of the subject.

In an embodiment, the finger slide control or the deployment trigger is configured to detach the nasal cavity device, the top stabilizer assembly, the stabilizer, or any combination thereof from the nasal applicator device such that the nasal applicator device can be removed from the nasal cavity and the nasal cavity device, the top stabilizer assembly, the stabilizer, or any combination thereof remains in the nasal cavity of the subject.

In an embodiment, the nasal applicator device further comprises a physical stopper attached to the nasal cavity device. In an embodiment the physical stopper is configured to mechanically deploy the top stabilizer assembly for an extended preferred placement of the nasal cavity device.

In an embodiment, the stabilizer is configured to be deployed at the opening of the olfactory cleft of the subject.

In an embodiment, the top stabilizer assembly comprises a rotatable or malleable arm. The rotatable or malleable arm may be useful in directing the nasal cavity device to the olfactory cleft of the subject.

In an embodiment, the rotatable arm is configured to be rotated with the one or more controls at an external angle of 45° to 90°.

In an embodiment, the stabilizer comprises a dried compressed sponge, an inflatable balloon, or any combination thereof.

In an embodiment, the dried compressed sponge has an inflated volume with contact with mucosal fluid. In this manner, the dried compressed sponge can be readily deployed to a desired position with minimal discomfort to the subject, and at the desired position come into contact with mucosal fluid to expand the sponge volume to maintain the desired position in the subject, optionally without any active deployment steps.

In an embodiment, the nasal applicator device comprises an inflatable balloon in operable connection with a controller to provide mechanical inflation having an inflated diameter of up to 6 mm.

In an embodiment, the nasal cavity device comprises an electrophysiological device having one or more electrodes at least partially disposed on a distal tip of the nasal cavity device.

In an embodiment, the nasal applicator arm distal end is configured to operably connect to the nasal cavity device.

Also disclosed herein is a method of electrophysiologically interfacing nasal cavity device with a biological tissue of a subject, the method comprising the steps of: providing a nasal cavity device as disclosed herein operably connecting a nasal cavity device with the applicator arm distal end to provide a loaded nasal applicator device; hand-positioning the handle to introduce the loaded nasal applicator device to a nasal cavity, including an anterior opening of the cribriform plate; first engaging the one or more controllers to advance the nasal cavity device between 10 mm to 40 mm so that at least a portion of the nasal cavity device is positioned within an olfactory cleft of the subject; deploying the stabilizer at the opening of an olfactory cleft of the subject so that at least a portion of a distal end of the nasal cavity device is in electrical contact with the sphenoid sinus wall; second engaging the one or more controllers to release the nasal cavity device and the stabilizer from the nasal applicator device; and removing the nasal applicator device from the nasal cavity, thereby electrophysiologically interfacing the nasal cavity device with the biological tissue.

Optionally, in an embodiment, the method comprises holding a nasal endoscope with a second hand to visualize a position of the nasal cavity device.

Optionally, in an embodiment, the method comprises simultaneously inserting the nasal applicator device and the nasal endoscope into the subject's nasal cavity.

Optionally, in an embodiment, the step of second engaging the one or more controllers further comprises: retracting the top stabilizer assembly from the applicator arm; and rotating the applicator channel cover; thereby releasing the top stabilizer assembly and the nasal cavity device from the nasal applicator device.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIGS. 1A-1C: Flexible Endoscopes. FIG. 1A: Basic components of standard flexible videoscope. FIG. 1B: Construction of bending section. FIG. 1C: Rotation of angulation knob caused deflection of bending section.

FIG. 2: Flexible endoscopic cameras.

FIG. 3: Top stabilizer—flex clip. Shows an exemplary embodiment of a nasal applicator device as disclosed herein. The nasal cavity device, shown as an electrode or an electrode array, is held within and stably immobilized by an applicator arm. The nasal applicator device of the embodiment shown comprises an applicator channel longitudinally aligned with the applicator arm. The applicator channel longitudinally extends from the handle distal end and is configured to connect to a nasal endoscope as shown which allows visualization of the nasal cavity enabling precision placement of a nasal cavity device, for example, an electrode. The top stabilizer assembly comprises a flex clip that is triggered by retraction of the release pin that is activatable in the handle (not shown). The flex clip is surrounded by expandable material like PVA foam which comprises the stabilizer.

FIG. 4: Top stabilizer—passive rotation/balloon. Shows an exemplary embodiment of a nasal applicator device as disclosed herein. The nasal cavity device, shown as an electrode or an electrode array, is held within and stably immobilized by an applicator arm. The nasal applicator device of the embodiment shown comprises an applicator channel longitudinally aligned with the applicator arm. The applicator channel longitudinally extends from the handle distal end and is configured to connect to a nasal endoscope which allows visualization of the nasal cavity enabling precision placement of a nasal cavity device, for example, an electrode. The top stabilizer comprises a balloon to which air is transferred from the handle of the nasal activator device to the balloon. The balloon then expands upward to secure the nasal cavity device.

FIG. 5: Exemplary embodiment of a nasal applicator device as disclosed herein. The nasal cavity device, shown as an electrode or an electrode array, is held within and stably immobilized by an applicator arm. The nasal applicator device handle comprises an advancement wheel to advance the electrode to the desired position. A second finger slide enables adjustment of the stabilizer such as a balloon. A release trigger enables deployment of the top stabilizer clip.

FIG. 6: Procedure for using a nasal applicator device as disclosed herein to insert a nasal cavity device.

FIG. 7: Procedure for using the nasal applicator device as disclosed herein to insert a nasal cavity device. The nasal cavity device is stably immobilized in the applicator arm of the nasal applicator device. The one or more controls are used to expand the balloon to the desired size, securing the stabilizer in place. The nasal cavity device or electrode is pushed forward using an advancement wheel. For example, the nasal applicator device with a nasal cavity device stably immobilized therein is inserted into the nasal cavity for placement of the nasal cavity device at the cribriform plate. Once the stabilizer, or top stabilizer is in position, the balloon expansion slide, or finger slide, is pushed forward expanding the balloon to the desired size, thus securing the stabilizer in position. The nasal cavity device, or electrode, is pushed forward using an advancement wheel.

FIG. 8: Procedure for using the nasal applicator device as disclosed herein to insert a nasal cavity device. The nasal cavity device is locked to the top stabilizer assembly by pulling back on the deployment trigger to a first position to release a spring clip which compresses onto the body of the electrode. The applicator arm is disconnected from the stabilizer by pulling the deployment trigger to a second position so that the connection arms spring inward.

FIG. 9: Applicator concept 1—procedure. Procedure for using the nasal applicator device as disclosed herein to release a nasal cavity device, here embodied as an electrode. The nasal cavity device is advanced through a closed guide channel.

However, when deployed in appropriate position, the guide channel can be opened with a rotating cover enabling the stabilized nasal device to be released into the nasal cavity. A balloon stabilizer can then be released from the nasal applicator device.

FIG. 10: Procedure for using the nasal applicator device as disclosed herein to release a nasal cavity device, here embodied as an electrode and a balloon for stabilization. A separate septum clip can help secure the nasal electrode. The electrode and the balloon remain in position. The septum clip may be secured to the electrode (not shown).

FIG. 11: Exemplary embodiment of a nasal applicator device as disclosed herein. Shown is a nasal applicator device with an applicator arm in which nasal cavity device is stably immobilized. The top stabilizer comprises a spring clip with PVA. One or more controls including an advance wheel and a and deployment trigger for top stabilizer release.

FIG. 12: Insertion of a nasal cavity device with a nasal applicator device as disclosed herein.

FIG. 13: Insertion of a nasal cavity device using an exemplary embodiment of a nasal applicator device as disclosed herein. Once the top stabilizer, or stabilizer is in the desired position, the PVA foam will begin to expand, securing the top stabilizer in position. The nasal cavity device, here an electrode, may be advanced by pressing down and rolling the advancement wheel forward. In the embodiment depicted, the nasal cavity device will bend forward due to the nasal cavity topography.

FIG. 14: Exemplary embodiment of a nasal applicator device as disclosed herein. the nasal applicator device comprises a spring clip. The nasal cavity device may be locked to the top stabilizer by pulling back on the trigger of the handle to release the spring clip so that it compresses onto the body of the nasal cavity device.

FIG. 15: Insertion of a nasal cavity device with a nasal applicator device as disclosed herein. The applicator arm comprises an electrode guide channel which is operated by a rotating cover.

FIGS. 16A-16B: Exemplary embodiments of two different devices

FIG. 17: Exemplary embodiment of a septum clip as disclosed herein.

FIG. 18: Exemplary embodiment of a septum clip as disclosed herein.

FIG. 19: Exemplary embodiment of a septum clip as disclosed herein.

FIG. 20: A detachable distal end effector that attaches to a nasal applicator device as disclosed herein with a curved distal guide channel for placement of a nasal device into the olfactory cleft. The distal end effector comprises a flex clip and a tip guide. The flex clip is interposed between the tip guide and the nasal applicator device to facilitate placement and removal.

FIG. 21: Exemplary embodiment of a detachable distal end effector comprising a tip guide and a flex clip.

FIG. 22: Exemplary embodiment of a detachable distal end effector comprising a tip guide and a flex clip.

FIG. 23: Anatomic illustration of the olfactory bulb and juxtaposition with the olfactory epithelium separated by the porous cribriform plate.

FIGS. 24A-24F: Placement procedure for using nasal applicator device to place a nasal device, shown as an electrode, in the olfactory cleft. FIG. 24A: Illustrates step 1: The nasal applicator device is placed into the nasal cavity until the tip guide, is aligned with the opening of the olfactory cleft. FIG. 24B: illustrates step 2: Using the advancement wheel, the nasal cavity device is advanced into the olfactory cleft. The PVA sponge surrounding the tip guide expands at the opening of the olfactory cleft to stabilize placement of the device. FIG. 24C: illustrates step 3: A release trigger that is located on the handle is activated when the electrode has reached the desired depth.

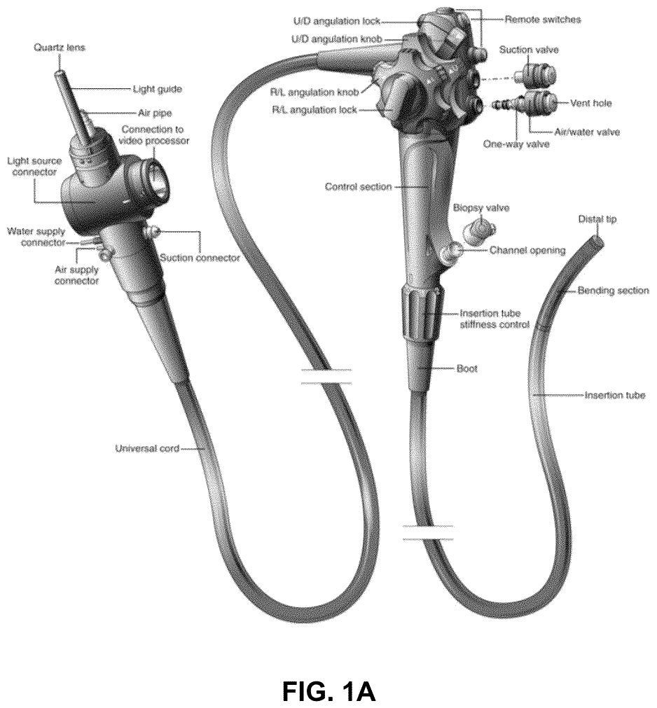

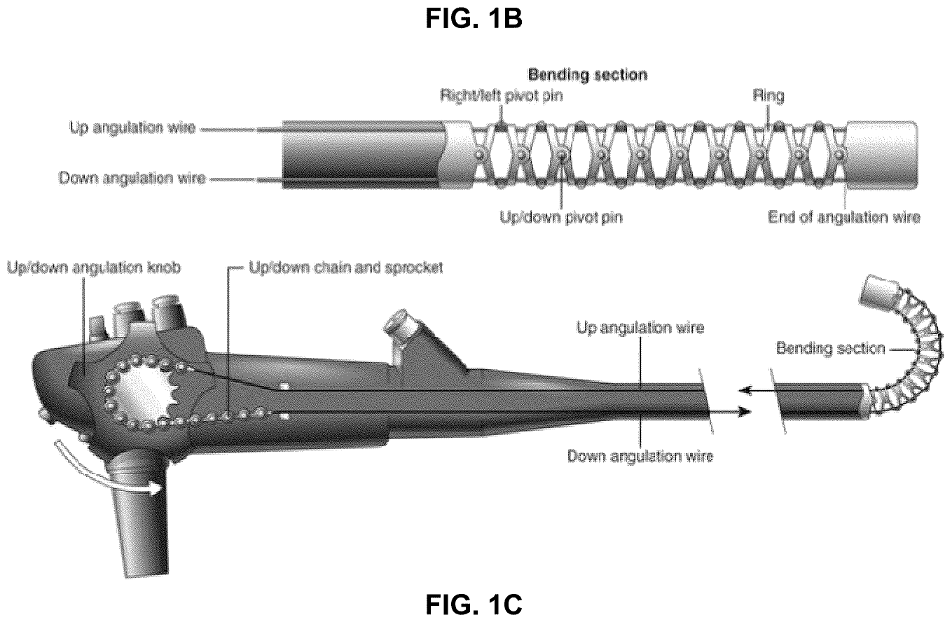

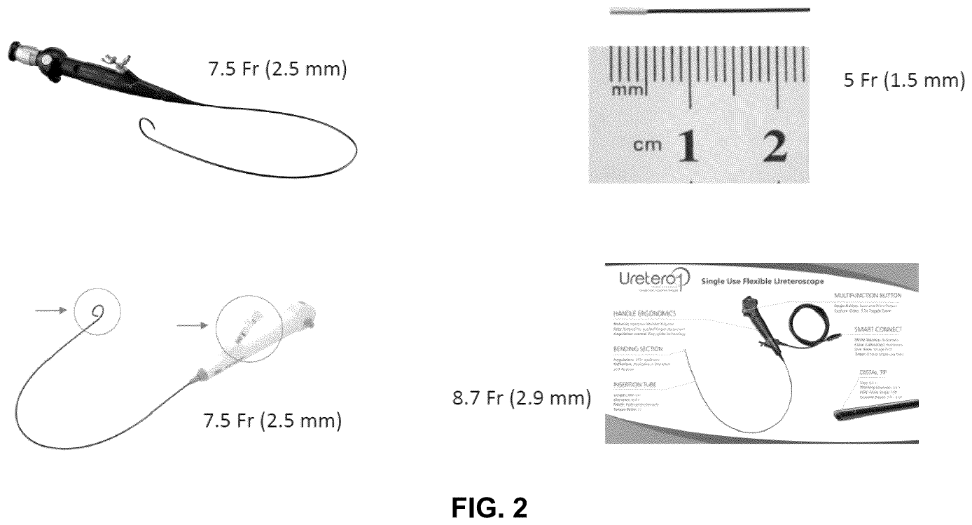

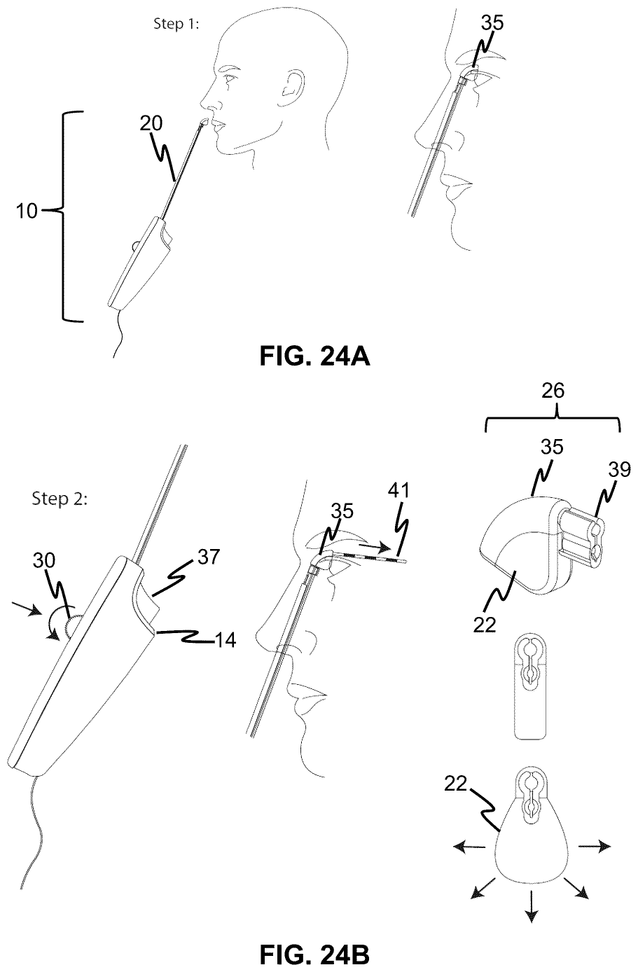

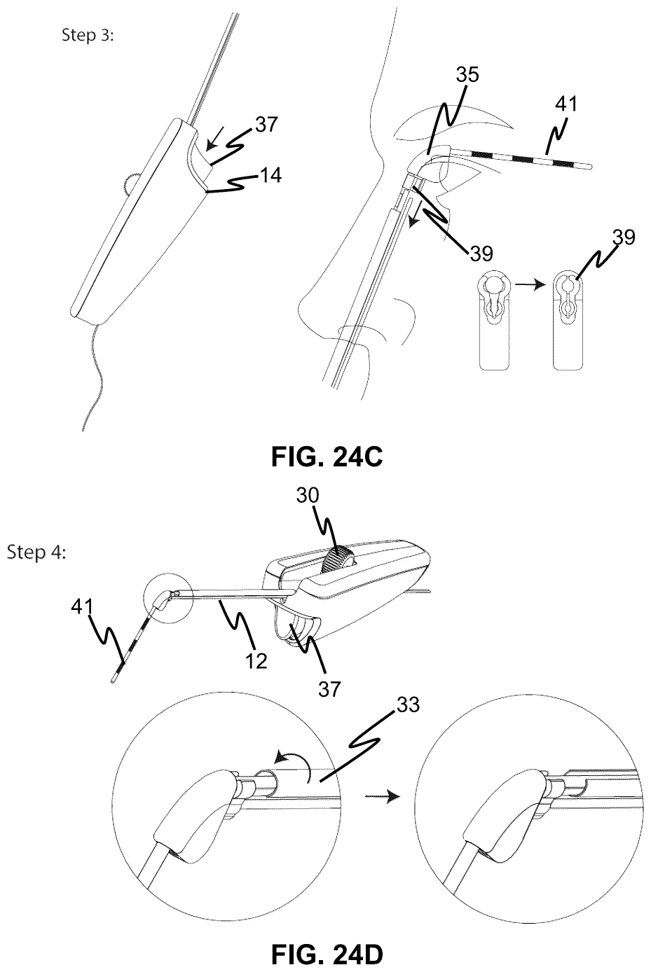

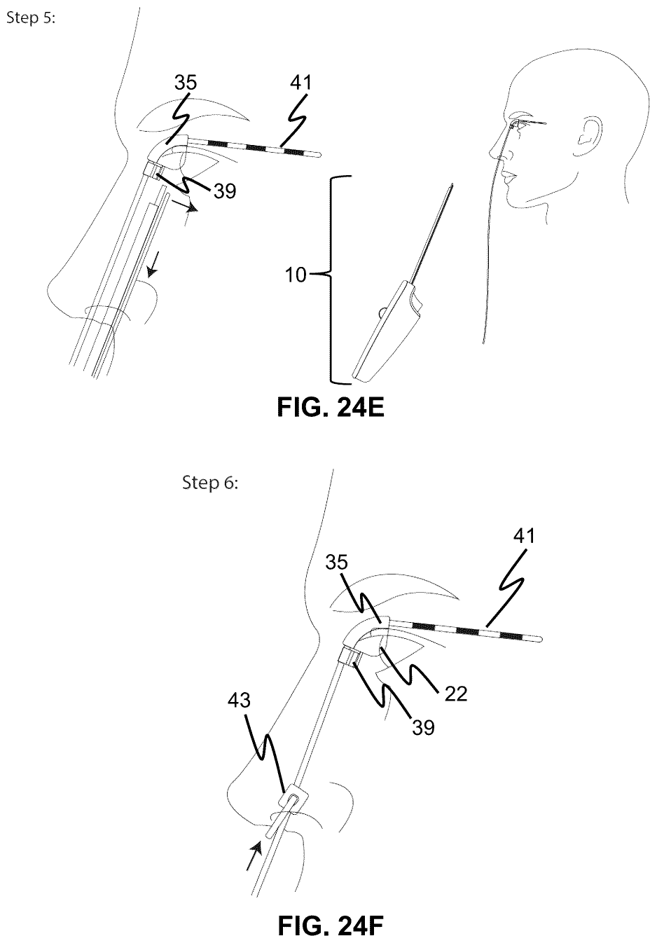

This retracts the top stabilizer release allowing the flex clip to grip on to the nasal cavity device and secures the flex clip to the tip guide. FIG. 24D: illustrates step 4: The release trigger also detaches the tip guide and rotates the applicator channel cover of the applicator arm to release the nasal device into the nasal cavity. FIG. 24E: illustrates step 5: The nasal applicator device, now released from the tip guide and flex clip is removed from the nasal cavity leaving the nasal cavity device in the olfactory cleft FIG. 24F: illustrates step 6: The septum clip is applied clamping the nasal cavity device to the nasal septum.

### DETAILED DESCRIPTION OF THE INVENTION

In general, the terms and phrases used herein have their art-recognized meaning, which can be found by reference to standard texts, journal references and contexts known to those skilled in the art. The following definitions are provided to clarify their specific use in the context of the invention.

The term “malleable” is used broadly herein to refer to a material that has the ability to bend, expand, contract, fold, unfold, or otherwise substantially deform or change shape under an applied force, without adversely impacting the material's functionality. A material that is more malleable than another is more able to change shape under an applied force without fracture or breaking. For example, a malleable refers to a component of the nasal applicator device that is able to bend such that the malleable arm conforms to an individual subject's nasal cavity anatomy, and that is capable of deforming as the device is fed along the nasal cavity.

The term “electrophysiological device,” as used herein, refers to an instrument designed to interface with a biological tissue, including to measure, record, modulate, or stimulate electrical activity in biological tissues or cells thereof. As used herein, electrophysiological device also refers to an instrument that may deliver an electrical signal to a biological tissue in the body. More particularly, an electrophysiological device refers to a device that measures or stimulates electrical signals in biological tissues.

Non-limiting examples of an electrophysiological device includes for example, an ultrasonic probe, one or more electrodes, a multi-contact electrode, an electrode strip, a directional-contact multi-contact electrode, an optical stimulator, a mechanical stimulator, a laser stimulator, an ultrasound stimulator, a magnetic stimulator, including a transcranial magnetic stimulator, a transcranial direct current stimulator, including a low-level laser and a transcranial laser, a transcranial alternating current stimulator, a fiber-optic probe, a magnetoencephalographic probe, or and any combination of these.

“Electrophysiologically interfacing” refers to the establishing of a functional connection between biological tissues and an electrophysiological device such as an ultrasonic probe, one or more electrodes, a multi-contact electrode, an electrode strip, a directional-contact multi-contact electrode, an optical stimulator, a mechanical stimulator, a laser stimulator, an ultrasound stimulator, a magnetic stimulator, including a transcranial magnetic stimulator, a transcranial direct current stimulator, including a low-level laser and a transcranial laser, a transcranial alternating current stimulator, a fiber-optic probe, a magnetoencephalographic probe, or and any combination of these. The interface allows for the exchange of electrical signals between the electrophysiological device and the biological tissue in electrical communication. Electrophysiologically interfacing enables, for example, the monitoring, recording, stimulation, or modulation of electrical activities within cells or biological tissues, facilitating electrical communication between biological tissues and electrophysiological devices.

The term “stabilizer,” as used herein refers to a material that stably maintains the positioning of a nasal cavity device of the present invention during use in a subject's nasal cavity. For example, the stabilizer may be a material that is able to expand upon positioning of the nasal cavity device in a subject's nasal cavity in order to enable a stable contact area between the nasal cavity device and an inner surface of the subject's nasal cavity. For example, a stabilizer may be used to secure the nasal cavity device between the subject's septal wall and middle turbinate. A stabilizer may be a material capable of expansion once the nasal cavity device has been properly placed within the subject's nasal cavity, preferably at a location between the subject's nasal wall and optionally the subject's lower, middle, or superior turbinates, including a sponge-like material. Additionally, the stabilizer may comprise a mechanical device, for example, a spring, a flap, a ring, a wedge, or u-shape, or any configuration of a mechanical device that may applies tension upon deployment, such as a spring-tension, or mechanical material-based tension. Useful materials for a mechanical stabilizer may comprise silicone, urethane, elastomer or other polymer. Useful materials for an absorptive stabilizer may comprise hydroxylated polyvinyl acetate, synthetic biodegradable fragmenting foam, oxidized nitrocellulose, cotton, non-woven gauze, polyether-polyurethane, polyurethane, medical-grade foam, silicone, polyvinyl acetate, a resorbable oxidized cellulose material (trade name: Surgicel), a thrombin soaked hemostatic device prepared purified porcine skin gelatin (trade name: Gelfoam), or a hemostatic matrix (trade name: Floseal).

As used herein, the term “nasal cavity device” is used interchangeably with the term “electrophysiological device” and refers to an instrument designed to interface with a biological tissue, including to measure, record, modulate, or stimulate electrical activity in biological tissues or cells thereof. As used herein, electrophysiological device also refers to an instrument that may deliver an electrical signal to a biological tissue in the body. More particularly, an electrophysiological device refers to a device that measures or stimulates electrical signals in biological tissues. Non-limiting examples of an electrophysiological device includes for example, an ultrasonic probe, one or more electrodes, a multi-contact electrode, an electrode strip, a directional-contact multi-contact electrode, an optical stimulator, a mechanical stimulator, a laser stimulator, an ultrasound stimulator, a magnetic stimulator, including a transcranial magnetic stimulator, a transcranial direct current stimulator, including a low-level laser and a transcranial laser, a transcranial alternating current stimulator, a fiber-optic probe, a magnetoencephalographic probe, or and any combination of these. Any of the nasal applicator devices described herein can be used with any of the nasal cavity devices described in PCT Application no. PCT/US2024/053078, filed Oct. 25, 2024, which is incorporated herein by reference for all purposes.

The term “biological tissue,” is used broadly herein, and includes any cells, tissue, or organs, including the skin or parts thereof, for example, mucosal tissues, epithelial tissues, and surfaces of the nasal cavity. The biological tissue can be in humans or other types of non-human animals (particularly mammals). Mammalian biological tissues directly or indirectly accessible from the nasal cavity include, for example, the olfactory and respiratory epithelium; trigeminal and olfactory nerves, cranial nerve zero, sympathetic nerves, parasympathetic nerves, olfactory bulb, olfactory tract, ventral brain, limbic brain, or any combination of these are of particular use with the present devices and methods.

As used herein, the term “operably connected” refers to a relationship between two or more components in which the components are mechanically, electrically, pneumatically, magnetically, or otherwise functionally coupled such that operation of one component influences, actuates, or controls the function, position, or state of the other. The connection may be direct or indirect, and may include intermediate components or linkages that transmit motion, force, signal, or control between the connected elements. In the context of the present disclosure, “operably connected” encompasses arrangements in which one component (e.g., a controller, handle, or applicator arm) is coupled to another component (e.g., a distal end effector or stabilizer) in a manner that enables coordinated movement or functional interaction during operation of the nasal applicator device. The connection need not be permanent, and may be removable, releasable, or re-engageable to facilitate assembly, sterilization, or repositioning of the nasal cavity device. In certain embodiments, the operative connection is provided by mechanical structures such as linkages, hinges, pivots, gears, cams, rods, shafts, threaded couplings, bayonet fittings, detents, snap-fit tabs, or frictional interfaces that transmit or translate motion or force. In other embodiments, the operative connection may include electrical wiring, conductive traces, magnetic couplings, pneumatic conduits, or hydraulic lines configured to convey signals or air between components.

As used herein, the term “stably immobilize” refers to a configuration or condition in which a component or assembly is maintained in a substantially fixed position or orientation relative to another structure so as to resist unintended movement, displacement, or rotation during operation or handling of the device as disclosed herein. The immobilization may be achieved through mechanical engagement, frictional retention, elastic deformation, adhesive forces, expansion of a material, or other stabilizing means, and is considered “stable” when the retained component remains functionally secured under expected procedural forces or manipulations. In the context of the present disclosure, “stably immobilize” encompasses arrangements in which the stabilizer maintains at least a portion of the nasal cavity device within the olfactory cleft in a manner that preserves its target position during use, as well as configurations in which a hollow tip or other element of the applicator arm retains the nasal cavity device within the applicator arm to prevent premature release or drift prior to placement. The term does not require complete rigidity or permanent fixation, but rather denotes sufficient positional stability to ensure accurate placement and controlled release of the nasal cavity device when desired. In certain embodiments, the immobilization is reversible, such that the retained component may be repositioned, released, or retrieved after installation without damage to the nasal cavity device or surrounding tissue.

As used herein, the term “releasably connected” refers to a connection between two components that is configured to maintain attachment during normal operation yet allow intentional separation without permanent deformation or damage to either component. The connection may be achieved by one or more mechanical engagement structures such as threads, bayonet fittings, keyed rotational couplings, snap-fit tabs, detents, latches, or frictional interfaces. In certain embodiments, release of the connection occurs via rotation of one component relative to the other, wherein the rotational motion acts upon the corresponding engagement structure, for example, by unthreading a threaded coupling, disengaging a bayonet slot, or rotating a keyed interface to an unlocked position, to separate the components. Accordingly releasably connected encompasses rotationally connected.

In the following description, numerous specific details of the devices, device components and methods of the present invention are set forth in order to provide a thorough explanation of the precise nature of the invention. It will be apparent, however, to those of skill in the art that the invention can be practiced without these specific details.

The invention can be further understood by the following non-limiting examples.

### EXAMPLE 1

### Customizable Configurations

In one embodiment, the system as disclosed herein includes a nasal applicator device which may comprise a top stabilizer, also referred to herein as a stabilizer. Also disclosed herein is a septum clip. Together, these components enable controlled installation, positioning, and stabilization of a nasal cavity device, for example, an electrode or electrode array, within the nasal cavity.

In embodiments, the nasal applicator device serves as a support structure for the electrode, or nasal cavity device, during installation and allows the installer to control the nasal cavity device, the top stabilizer, and the visualization method during the placement procedure. The nasal applicator device may incorporate a depth or position indicator to assist with accurate placement and may include a holder, or an applicator channel, for an endoscope camera to provide visualization of the procedure in real time. In certain configurations, the nasal applicator device is further configured to control the deployment or retraction of the top stabilizer mechanism. The nasal applicator device may be disengaged from the nasal cavity device and withdrawn from the nasal cavity without disturbing the position of the nasal cavity device, and can be re-engaged if repositioning of the nasal cavity device is needed. In some embodiments, the nasal applicator device includes a controller, such as a finger slide, advancement wheel, or other actuator, that enables controlled advancement of the electrode by a defined distance, for example approximately 35 mm, typically within a range of 10 to 40 mm. The distal end of the nasal applicator device may include a shaped or bent tip, for example a tip guide as disclosed elsewhere herein, configured to guide the nasal cavity device along a desired path and prevent contact with the roof of the nasal cavity during insertion. In certain designs, the applicator may eliminate the need for a separate stylus.

The top stabilizer, or stabilizer, is configured to secure the nasal cavity device in position along its length, typically after the bend in the nasal cavity device or at the opening of the olfactory cleft. The stabilizer is designed so that it does not obstruct visualization by the endoscope camera and can be adjusted to the desired location before being locked in place. The connection between the stabilizer and the nasal cavity device may be kept as compact as possible to minimize obstruction and tissue contact. In one implementation, the stabilizer includes an expandable element, such as a moisture-expanding PVA foam or another material that expands to hold the nasal cavity device securely against surrounding tissue and collapses back to its original size for repositioning or removal. The stabilizer is designed not to block airflow through the nasal passage and to minimize irritation by limiting contact with surrounding tissue. When deployed, the stabilizer maintains the nasal cavity device body against the upper surface of the nasal channel, thereby stabilizing its position during the procedure.

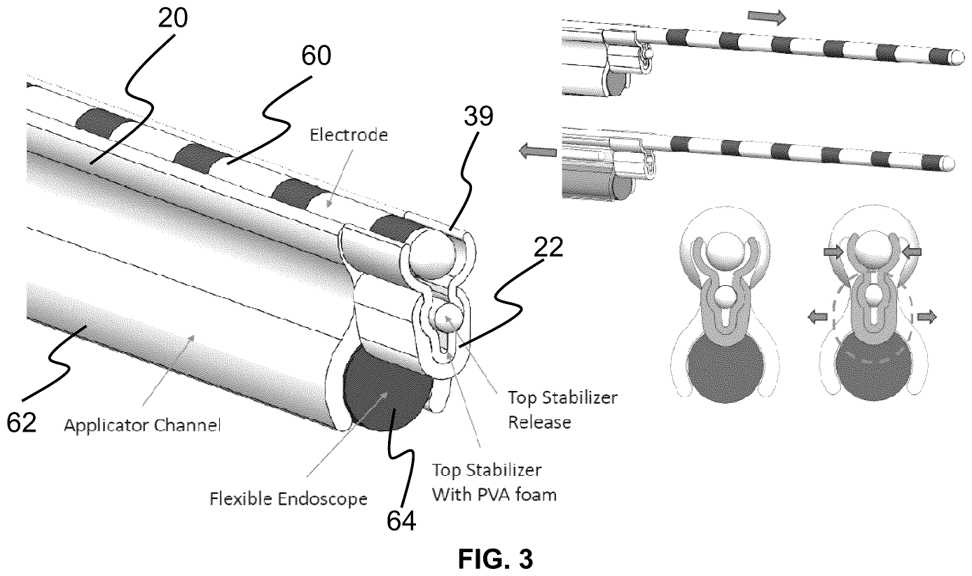

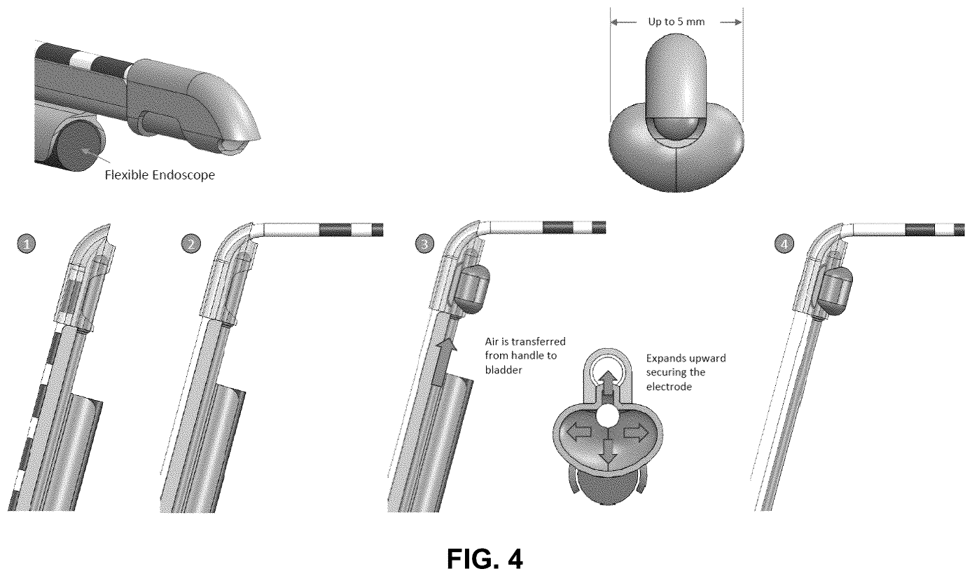

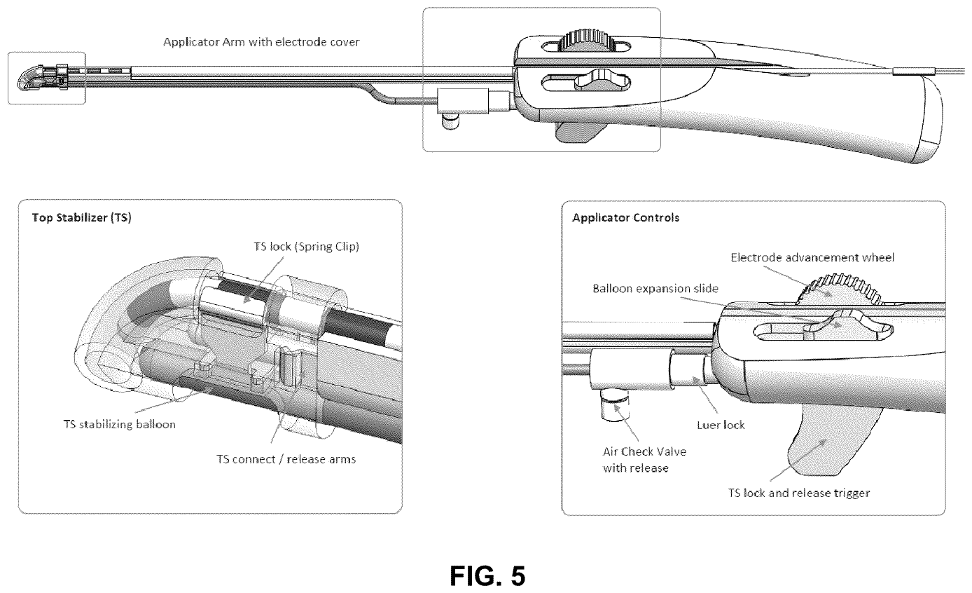

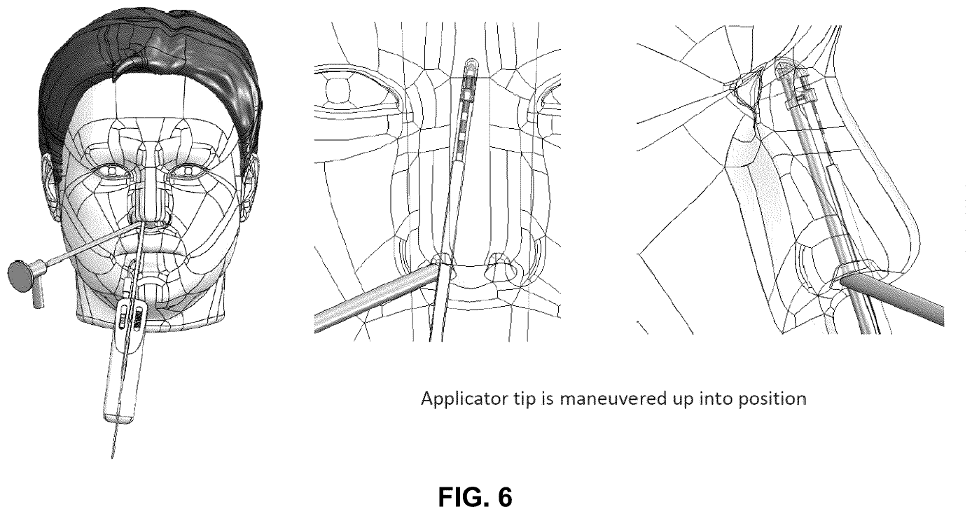

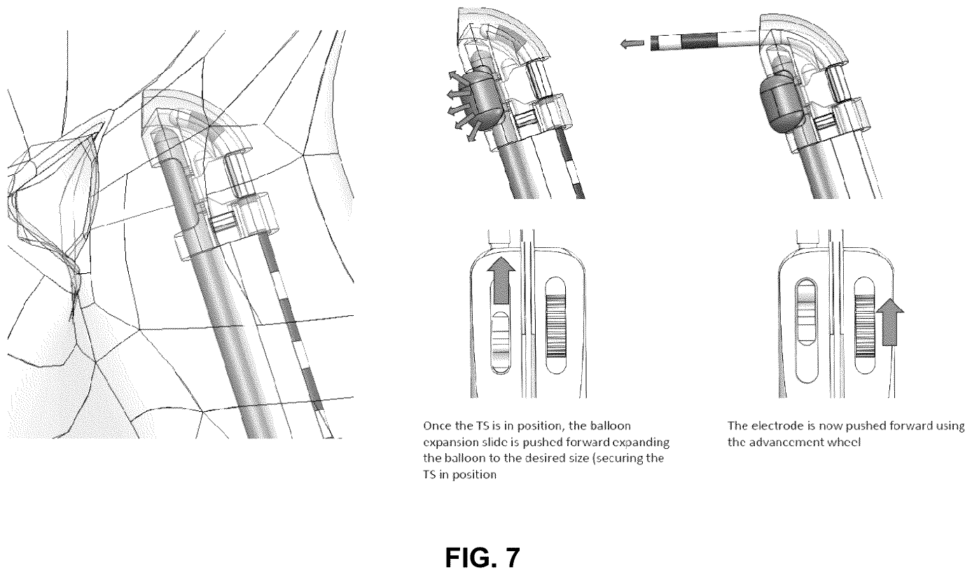

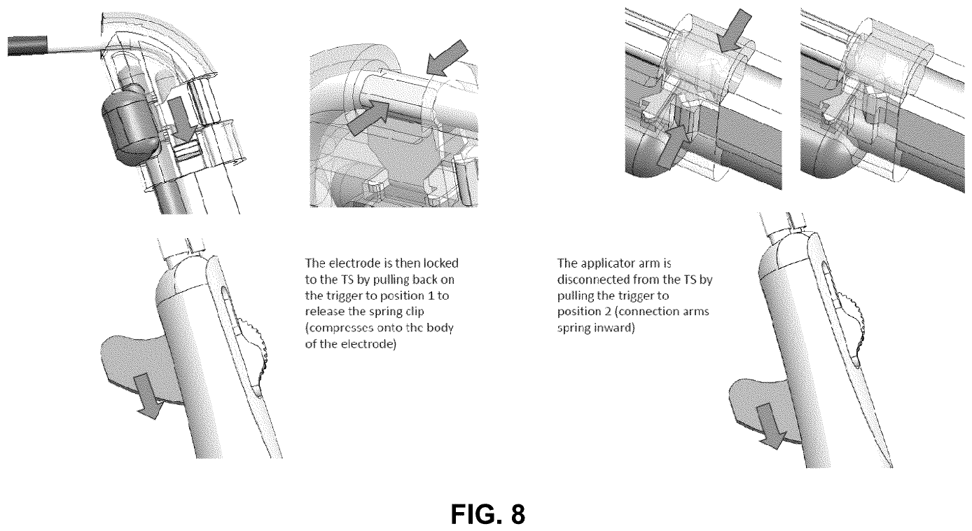

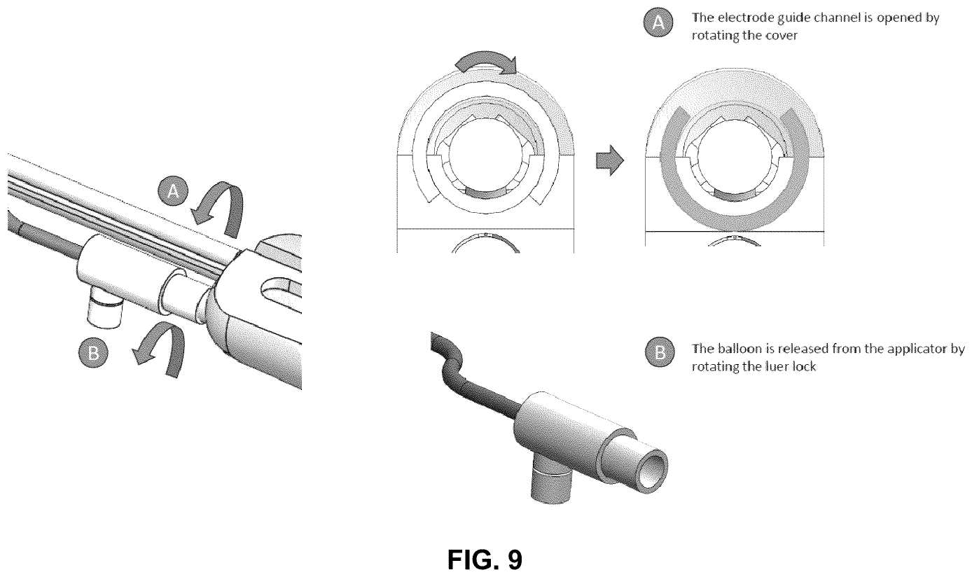

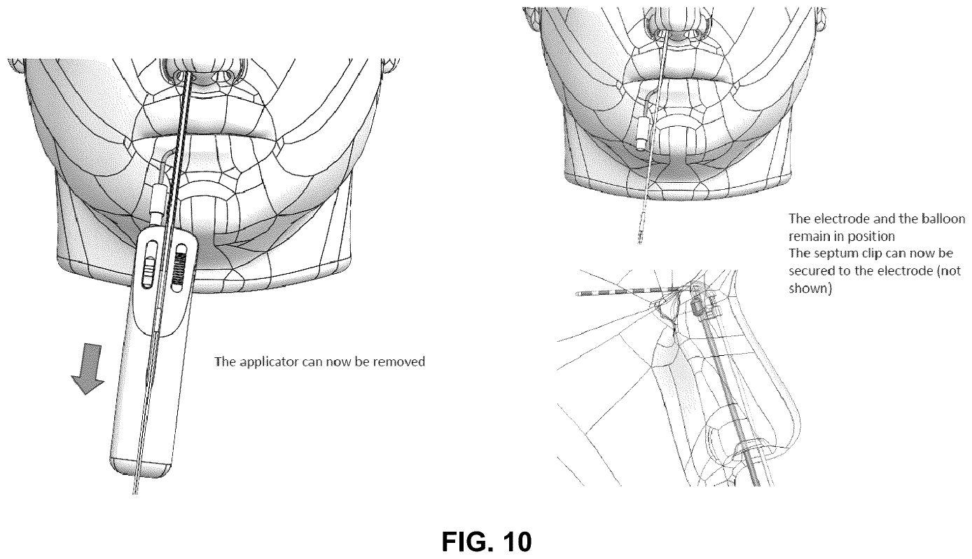

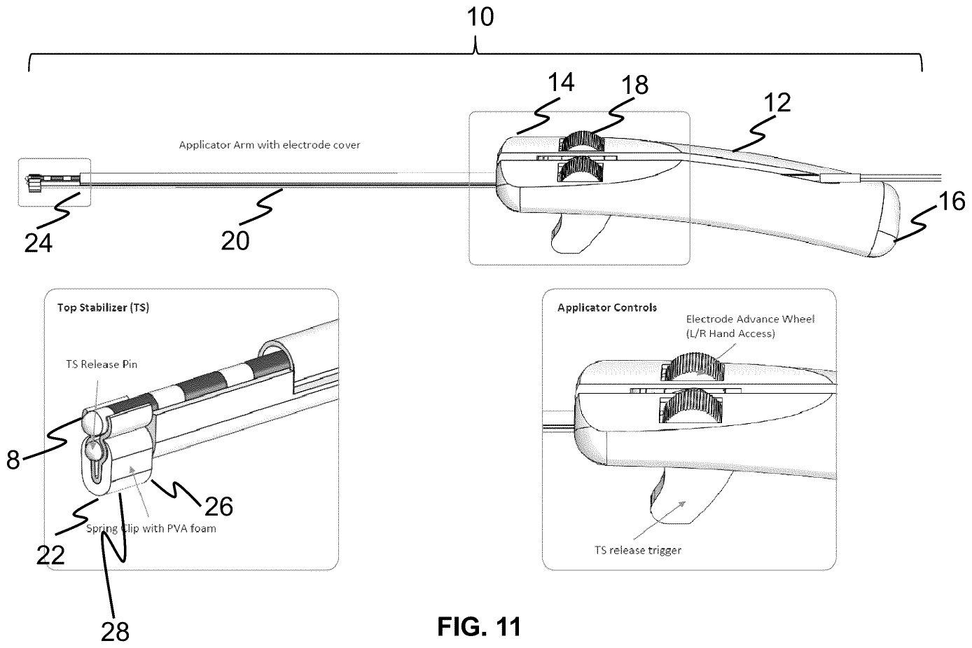

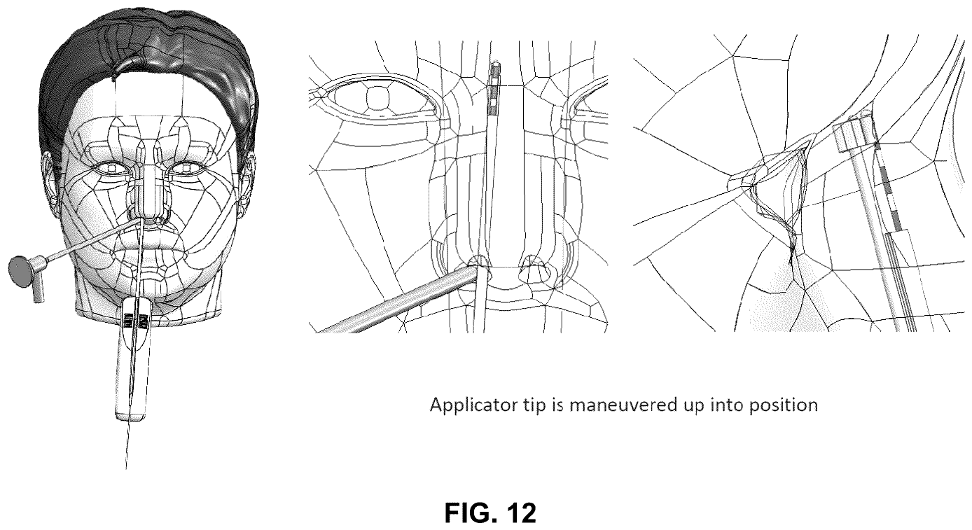

FIG. 3 shows an exemplary embodiment of a nasal applicator device. The nasal cavity device, shown as an electrode 60 or an electrode array, is held within and stably immobilized by an applicator arm 20. The nasal applicator device of the embodiment shown comprises an applicator channel 62 longitudinally aligned with the applicator arm. The applicator channel longitudinally extends from the handle distal end and is configured to connect to a nasal endoscope 64 which allows visualization of the nasal cavity enabling precision placement of a nasal cavity device, for example, an electrode. The top stabilizer assembly comprises a flex clip 39 that is triggered by retraction of the release pin that is activatable in the handle. The flex clip 39 is surrounded by the stabilizer 22, such as expandable material like PVA foam.

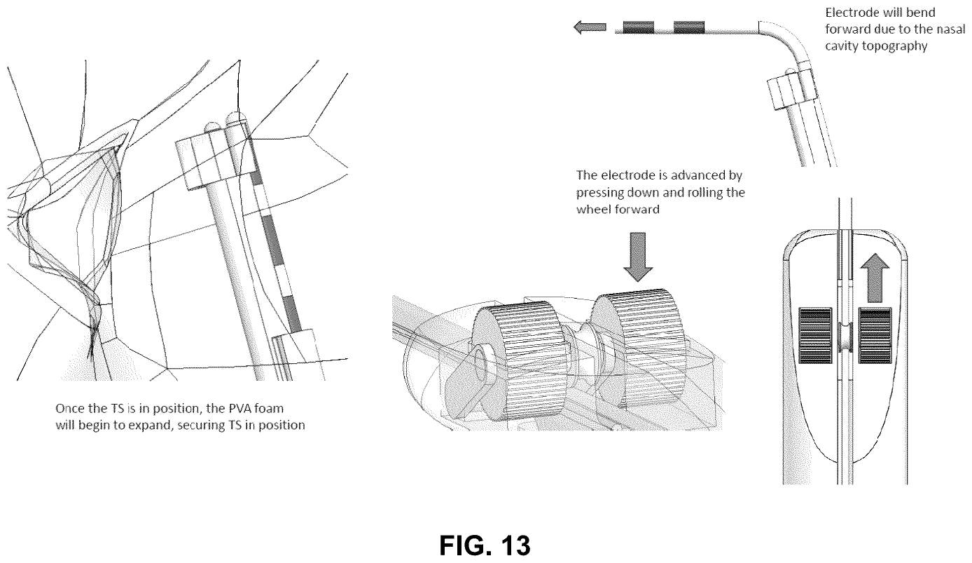

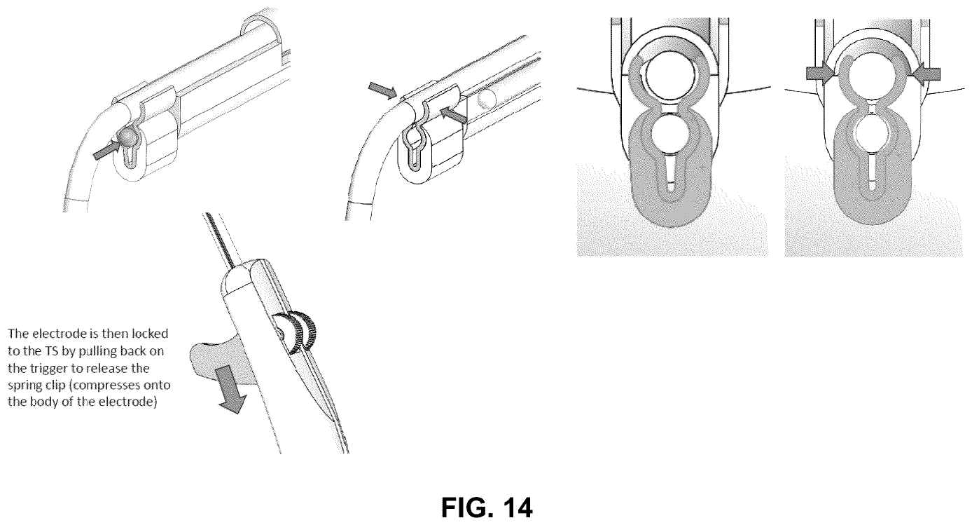

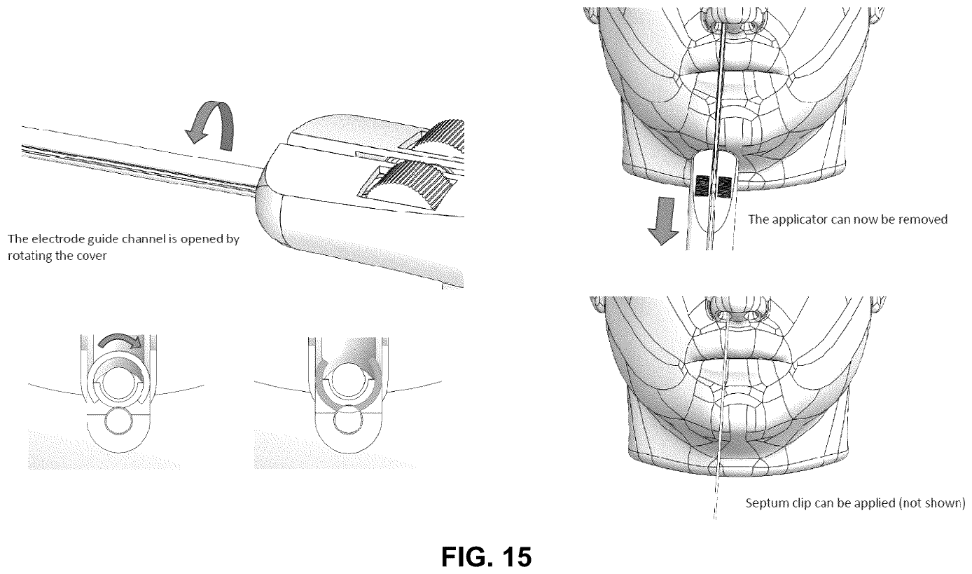

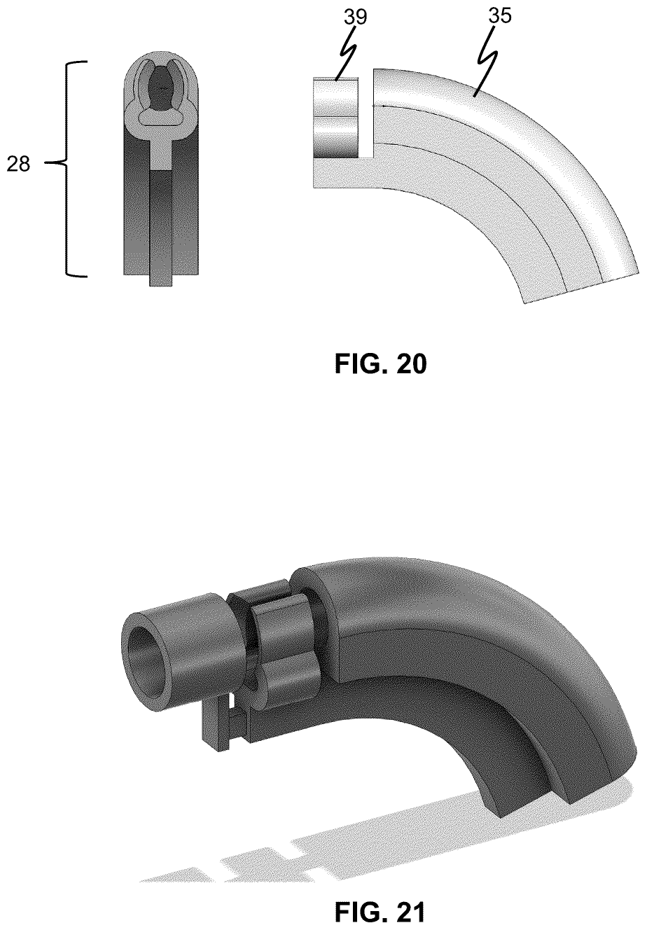

FIG. 13 shows an exemplary embodiment of a nasal applicator device 10 as disclosed herein. The nasal applicator device 10 allows for precision placement of a nasal cavity device in an olfactory cleft of a subject. The nasal applicator device 10 comprises a handle 12 extending between a handle distal end 14 and a distal proximal end 16; one or more controllers 18 operably connected to the handle; an applicator arm 20 connected to the handle distal end 14 a distal end effector 28 optionally comprising a tip guide 35 and a flex clip 39, for example, as shown in FIGS. 20-22, configured to operably connect to the applicator arm 20 to guide placement of the nasal cavity device; a stabilizer 22 operably connected to an applicator arm distal end 24 wherein during use the stabilizer is configured to stably immobilize at least a portion of the nasal cavity device within the olfactory cleft of the subject. Optionally, the stabilizer 22 is part of a top stabilizer assembly 26, the top stabilizer assembly comprising the stabilizer, the tip guide, and the flex clip.

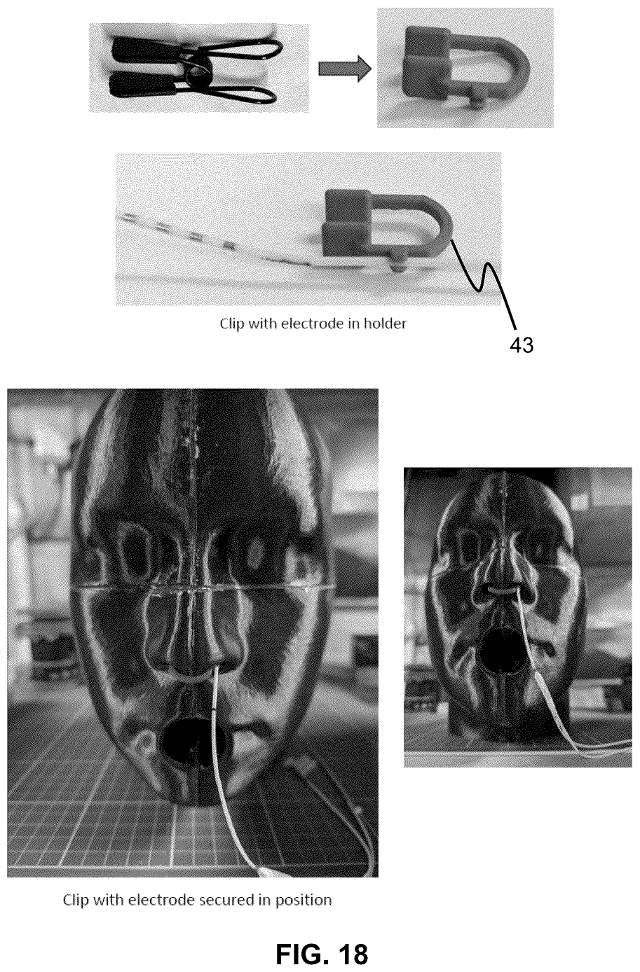

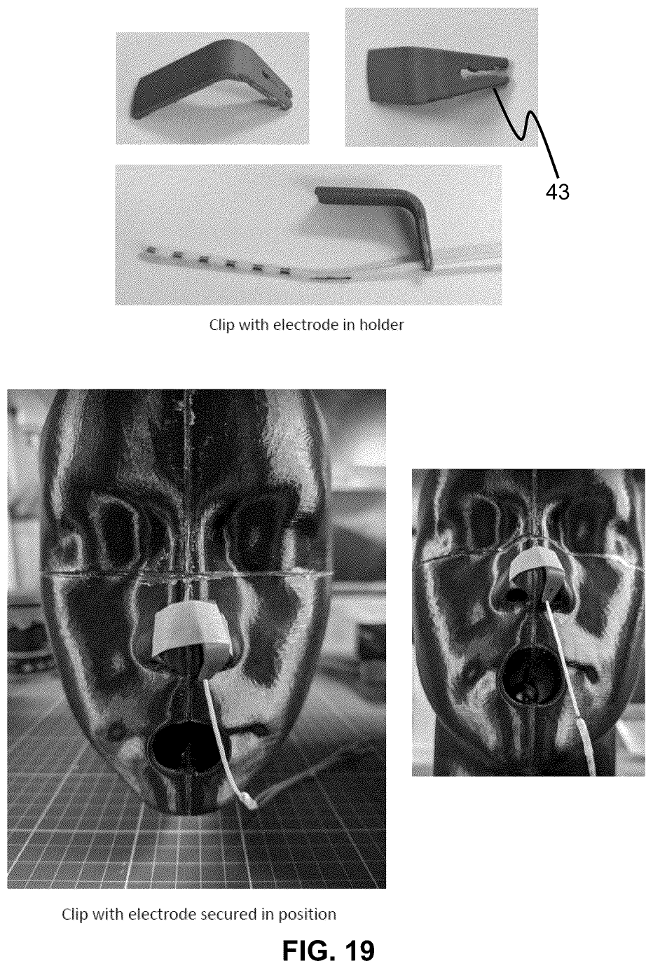

A septum clip 43 may be configured to secure the nasal cavity device near the tip of the nose along the septum as shown in FIG. 18 and FIG. 19. The septum clip 43 provides sufficient retention to maintain electrode placement during normal patient movement but allows for removal when desired. It is designed to avoid interference with normal breathing and does not function as a nasal dilator. The clip applies minimal compression force to reduce irritation or discomfort to the patient and is configured so as not to interfere with secondary air supply tubing if present. In use, the septum clip maintains electrode stability for monitoring sessions that may range from approximately twenty minutes to six hours.

### Key Operational Challenges and Design Approaches

In some embodiments, various operational challenges are addressed through specific design features and functional mechanisms, as summarized below. These challenges include determining accurate electrode placement, guiding the electrode around anatomical contours, securing and engaging the top stabilizer, removing the applicator without disturbing electrode position, and enabling re-engagement for repositioning.

A first operational challenge involves determining the electrode placement below the olfactory nerve without requiring a CT scan. In one approach, the nasal applicator device may use an endoscope to visualize the nasal topography, optionally with an integrated or secondary measurement system, such as a mechanical ruler or a laser projection system, to estimate depth and positioning. Alternatively, an electrode, or other nasal cavity device, having a sufficient number of contacts may be used to span the expected positional tolerance range. In such cases, placement can be determined based on the position of the top stabilizer, or stabilizer, which remains visible through the endoscope during installation.

A second challenge involves feeding the electrode around the corner within the nasal passage and advancing it into its final position. To address this, the system may include one or more mechanisms for pushing the electrode forward, such as a friction roller or similar drive component. Additional mechanisms may be incorporated to bend or direct the electrode as it is being advanced. The bending action may be active, for example by enabling rotation of a component or adjustment of the top stabilizer prior to forward advancement, or passive, relying on a predefined curvature integrated into the top stabilizer assembly housing to guide the electrode along the nasal contour.

A third challenge relates to securing the stabilizer, and/or the top stabilizer assembly, onto the electrode once the electrode is in place. Various mechanisms, as disclosed herein, may be deployed for this function, including a spring clip mechanism, a bladder or balloon-based mechanism (either custom or off-the-shelf), or a custom expanding pad or arm mechanism configured to apply gentle pressure around the electrode body.

A fourth challenge involves engaging the stabilizer once positioned within the nasal cavity. Several options are available depending on material selection and procedural requirements. For example, the stabilizer may employ PVA foam that expands upon moisture exposure, or a balloon or bladder-based mechanism that can be inflated after placement, or a custom expanding pad or arm mechanism capable of controlled deployment to stabilize the electrode against the surrounding tissue.

A fifth operational challenge concerns removal of the applicator without dislodging the electrode. In some embodiments, the applicator is detachably or releasably connected to the top stabilizer, allowing the applicator to be released and withdrawn while leaving the electrode and stabilizer undisturbed. Once detached, the nasal applicator device may simply slide backward along the nasal passage and exit through the nostril.

A sixth challenge involves enabling re-engagement of the electrode and stabilizer and/or top stabilizer assembly to allow repositioning if required. This may be accomplished by sliding the applicator back into the nasal passage along the electrode body to re-engage the stabilizer and/or top stabilizer assembly. The applicator may then be used to collapse or deflate the stabilizer, allowing the electrode position to be adjusted. Once the desired placement has been reestablished, the stabilizer can be re-inflated or expanded to secure the electrode in its new position.

### EXAMPLE 2

### Alternative Electrode Placement and Stabilization Embodiments

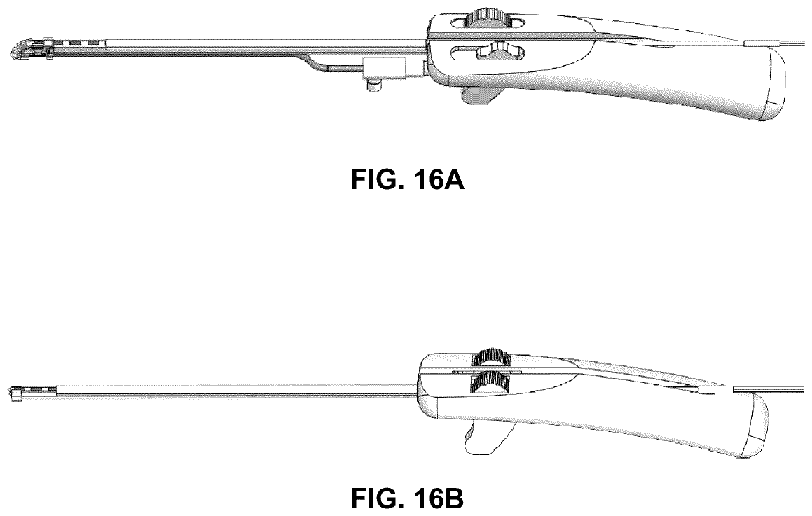

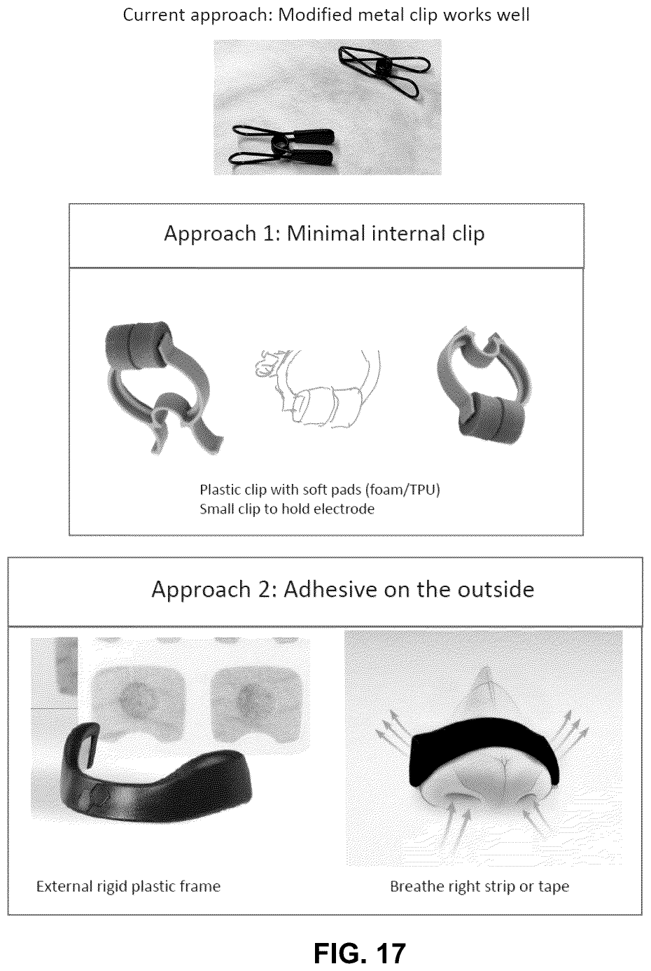

In the embodiment illustrated in FIG. 16A, the electrode placement strategy is based on pre-procedure imaging and top stabilizer positioning. Specifically, electrode placement may be guided using a CT scan, and the final position of the electrode is dependent upon the stabilizer and/or top stabilizer assembly, which is secured to the electrode at the end of the contact region. The contact region may be dimensioned such that its length accommodates the full expected tolerance range for placement within the nasal cavity.

During installation, visualization may be achieved using an endoscope, and the nasal applicator device is operated by the installer with the other hand to manipulate and advance the electrode. Electrode corner bending may be integrated into the housing of the top stabilizer, allowing the electrode to conform to the desired anatomical pathway during deployment.

Locking of the stabilizer and/or the top stabilizer assembly to the electrode may be accomplished through a spring-clip mechanism that provides secure engagement while allowing for controlled release when needed. To secure the stabilizer in position within the nasal cavity, a balloon element may be employed. The balloon can be inflated to apply gentle pressure against the surrounding tissue, thereby stabilizing the electrode in the desired position.

In certain embodiments, the system is configured to enable repositioning of the top stabilizer after installation. The balloon can be reattached to the nasal applicator device to contract, allowing partial withdrawal of the stabilizer. The applicator may then reengage with the stabilizer, and the installation process can be repeated using the same stabilizer without removal of the entire system.

From a patient comfort perspective, the top stabilizer in this embodiment is larger overall and may therefore be perceptible to the patient; however, the balloon surface is less abrasive than PVA foam, making removal and repositioning less traumatic. Both the electrode and the balloon air line may exit through the nostril for connection to external controls.

Installation of this system typically involves nine steps, reflecting its relatively high system complexity. Several mechanisms incorporated in this configuration may require evaluation and refinement, and the system design demands higher user coordination due to the increased number of procedural steps and control elements.

In the embodiment illustrated in FIG. 16B, the electrode placement strategy relies on nasal cavity topography rather than pre-procedure imaging. Placement is determined primarily by the stabilizer and/or top stabilizer assembly, which is positioned along the electrode at the distal end of the contact region.

Visualization during installation is again provided by an endoscope, but the process is simplified, with the nasal applicator device operated using one hand and fewer interdependent mechanisms overall. In this configuration, electrode corner bending is achieved by conformance to the natural topography of the nasal cavity, rather than by a pre-formed bend integrated into the stabilizer housing.

The stabilizer and/or top stabilizer assembly is locked to the electrode using a spring-clip mechanism, similar to the embodiment of FIG. 16A, but the top stabilizer assembly is secured in position by a PVA foam element rather than a balloon. The PVA foam expands upon moisture absorption to hold the electrode against the surrounding tissue.

Repositioning after installation is more limited in this embodiment. To adjust or replace the stabilizer and/or the top stabilizer assembly must typically be manually removed. The stabilizer is separated from the electrode, and a new stabilizer is applied, after which the system may be reinstalled.

In terms of patient comfort, the PVA foam stabilizer may be more abrasive during removal compared with the balloon-based design of FIG. 16A. However, the smaller overall size of the stabilizer may reduce the sensation of pressure within the nasal cavity during use.

Installation using the embodiment depicted FIG. 16B generally requires five steps, resulting in lower system complexity.

As shown in FIG. 19, a septum clip 43 embodiment is configured with a pad size similar to that of a metal spring clip while reducing obstructions below the nose. The clip is further configured to attach directly to an electrode. Installation involves sliding the septum clip into position, positioning the electrode, and then pushing the electrode into the septum clip to achieve engagement. In certain embodiments, one or more foam pads may be added to enhance comfort and accommodate a wider range of patient anatomies.

As also shown in FIG. 19, a second septum clip 43 embodiment is configured to utilize an external adhesive tape—such as a Breathe Right® strip—to secure the septum clip to the patient's nose. This configuration reduces obstructions both below and within the nasal region and is designed to provide improved long-term comfort by minimizing pressure on sensitive areas. The clip is further configured to attach directly to an electrode, or nasal cavity device as disclosed elsewhere herein. Installation involves applying the adhesive tape to the plastic clip, positioning the clip on the patient, placing the electrode, and pushing the electrode into the septum clip to secure it.

### EXAMPLE 3

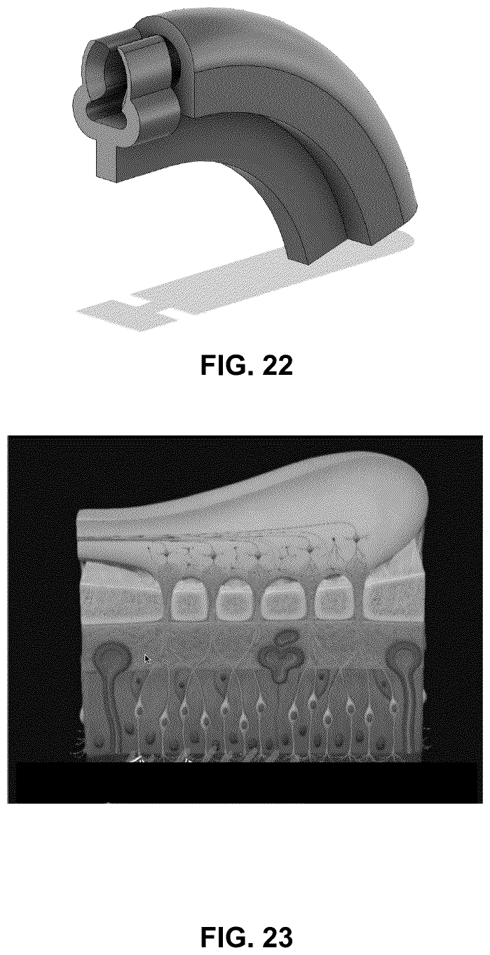

FIG. 20 shows an exemplary embodiment a distal end effector 28 comprising a tip guide 35 and flex clip 39 assembly for attachment to a nasal applicator device 10 as disclosed herein. This tip guide 35 allows for precision placement of a nasal cavity device into an olfactory cleft of a subject. The tip guide 35 is a channeled, assembly and an attached flex clip 39 that may be deployed by the activation of the applicator trigger. The juxtaposition of the flex clip 39 and the tip guide 35 and/or stabilizer may be embodied in FIG. 21 or FIG. 22. The purpose of the tip guide is to place the nasal device in the olfactory cleft immediately under the olfactory bulb which is depicted in FIG. 23.

### EXAMPLE 4

### A Device for Placement of a Nasal Cavity Device

Prior work has produced a method for recording signals and stimulating tissue at the human nasal cleft using a nasal cavity device, for example, an intranasal electrode, as described in PCT Application no. PCT/US2024/053078, which is incorporated by reference herein for all purposes, and specifically for any devices or nasal cavity devices disclosed therein that can be used with the instant nasal applicator devices to reliably and efficiently position a nasal cavity device. However, placement of such a nasal cavity device, for example, an intranasal electrode, is difficult, creating a need for a better method of placement. Here, we describe a nasal applicator device for placement of a nasal cavity device, for example, an intranasal electrode in humans, along with an explanation of the need for such a device. Accordingly, any of the nasal applicator devices described herein can be used with any of the nasal cavity devices described in PCT Application no. PCT/US2024/053078, filed Oct. 25, 2024, which is incorporated herein by reference for all purposes.

In an embodiment, disclosed herein is a method of electrophysiologically interfacing a nasal cavity device with a biological tissue of a subject, the method comprising the steps of: providing a nasal applicator device as described elsewhere herein; operably connecting a nasal cavity device with the applicator arm distal end to provide a loaded nasal applicator device; hand-positioning the handle to introduce the loaded nasal applicator device to a nasal cavity, including an anterior opening of the cribriform plate; first engaging the one or more controllers to advance the nasal cavity device between 10 mm to 40 mm so that at least a portion of the nasal cavity device is positioned within an olfactory cleft of the subject; deploying the stabilizer at the opening of an olfactory cleft of the subject so that at least a portion of a distal end of the nasal cavity device is in electrical contact with the sphenoid sinus wall; second engaging the one or more controllers to release the nasal cavity device and the stabilizer from the nasal applicator device; and removing the nasal applicator device from the nasal cavity, thereby electrophysiologically interfacing the nasal cavity device with the biological tissue.

The study and treatment of human olfaction and its pathologies has been hampered by the difficulty of direct imaging or recording of the primary olfactory peripheral and central structures: the olfactory epithelium and the olfactory bulb. These structures are located at the top of the nasal cavities, with the olfactory epithelium concentrated in the dorsal aspect of the olfactory cleft, and the olfactory bulb approximately 1-2 mm above it, with the two separated by a thin perforated section of the skull called the cribriform plate, through which the olfactory nerves connect the two structures. These structures are the first areas of odor processing and understanding them is important to understanding the human sense of smell. By placing a nasal cavity device, for example, an intranasal electrode—a very thin wire with electrical contacts along its tip—up into the olfactory cleft, one can record signals from both the epithelium and the bulb. These signals are produced during the sensing and processing of odors, and they provide a wealth of data that can be used to understand the system in health and disease, allowing for diagnostics and interventions in olfactory disease. However, placement of a nasal cavity device, for example, an intranasal electrode, at this location is technically challenging. This area of the nose is rarely accessed, even by ENTs, except during skull-base surgery when it is typically destroyed to allow access to the brain (to remove tumors, for example) and instrumentation to access this area in an awake patient is lacking. The nasal cavity device, for example, an electrode, must be precisely placed in a very small area that is unfamiliar to common ENT practice. In order to place a nasal cavity device, the ENT must maneuver a nasal endoscope in one hand, and carefully navigate a tortuous nasal cavity with the nasal cavity device held by a clamp in the other hand, then place and release the nasal cavity device adjacent to the cribriform plate, and remove the clamp without moving the nasal cavity device from its desired placement. The nasal cavity device can be floppy and otherwise difficult to handle, resulting in inexact placement, or can require adjustment. Furthermore, placement through the nasal cavity does not naturally follow the trajectory of the cribriform plate. Contact of the nasal cavity device and placement device with parts of the nasal cavity are very sensitive, so adjustments can be painful or stimulate mucus production that degrades electrophysiologic recordings.

What is needed is a method to place the nasal cavity device that improves ease of placement, accuracy of placement and safety of the placement procedure. Such a nasal applicator device reduces the training burden on clinicians who wish to use this technology, would increase safety of the use of this technology, and increase accuracy and speed in clinical applications. As a result, such a nasal applicator device allows broader use of the method, which confers the clinical benefits of the method to a broader clinical population. The requirements for such a nasal applicator device include that it be operable with one hand, that it allow fine-tune adjustment with that one hand, that the distal end of the nasal applicator device would be thin enough to fit into the upper nasal cavity while holding the nasal cavity device (FIG. 24A), that the distal end of the nasal applicator device be strong enough to hold and deploy the nasal cavity device with precision. The nasal applicator device may be operable with one hand because the clinician's other hand may be occupied during placement by a video-enabled endoscope, which is required to allow the clinician visual navigation of the device through the nasal cavity. All adjustments needed during nasal cavity device placement (listed below) may be one-handed as well, for the same reason. The placement device, also referred to as the nasal applicator device, should also have the capability of varying placement across individuals to account for individual anatomy using flexible materials such as a sponge and allow the clinician to advance the nasal cavity device along the nasal applicator device for a more distal placement, or retract the nasal cavity device along the nasal applicator device for a more proximal placement (FIG. 24B). In addition, such a nasal applicator device is able to firmly hold the nasal cavity device (FIG. 24C), giving the clinician precise control as they maneuver the nasal cavity device into place, but they would be able to deploy the nasal cavity device by releasing it in such a manner that the nasal cavity device remains in place (FIG. 24D), and the nasal applicator device may be maneuvered out of the nasal cavity without disturbing the now placed nasal cavity device (FIG. 24E). Additional stabilization of the nasal cavity device can be achieved using separate stabilization mechanisms like a nasal septal clip (FIG. 24F) The design contained herein meets all of these requirements.

The devices and methods disclosed herein solves these problems by allowing one-handed control over placement, bending the nasal cavity device so it conforms to the direction of the olfactory cleft, stabilizes the nasal cavity device at the desired placement, and affords customizability of placement length without obstructing the nasal passage.

The devices and methods disclosed herein solves the lack of suitable instrumentation for accessing and placing nasal cavity devices into the human olfactory cleft. This has prevented fundamental understanding of human olfactory disorders and prevented treatment of human olfactory disorders using direct electrical stimulation.

### EXAMPLE 5

FIGS. 24A-24F illustrate an exemplary embodiment of a nasal applicator device and an exemplary method of using the nasal applicator device to insert a nasal cavity device into the olfactory cleft of a subject. FIG. 24A: Illustrates step 1 the method: the applicator arm 20 of the nasal applicator device 10, is used to position at least a portion of the nasal cavity device into a subject's nasal cavity, including until the tip guide 35 is aligned with the opening of the olfactory cleft. FIG. 24B: illustrates step 2: Using a controller, illustrated as an advancement wheel 30, the nasal cavity device 41 is advanced into the olfactory cleft. The stabilizer 22, in this embodiment a PVA sponge surrounding the tip guide 35, expands at the opening of the olfactory cleft to stabilize placement of the nasal cavity device. FIG. 24B also illustrates a release trigger, also referred to herein as a deployment trigger 37 which is provided on the handle distal end 14. Also shown is a top stabilizer assembly 26. The top stabilizer assembly in the embodiment shown comprises the tip guide 35, the flex clip 39, and the stabilizer 22. FIG. 24C: illustrates step 3: A release trigger, also referred to herein as a deployment trigger 37, that is located on the handle distal end 14 is activated, for example manually activated, when the nasal cavity device 41 has reached the desired depth in the olfactory cleft, for example, 10 mm to 40 mm depending on the subject's anatomy. This retracts the top stabilizer assembly release causing the flex clip 39 to retract as shown and to grip on to the nasal cavity device 41 and secures the flex clip 39 to the tip guide 37. FIG. 24D: illustrates step 4: The release trigger 37 also detaches the tip guide 35 and rotates the applicator channel cover 33 of the applicator arm 12 to release the nasal cavity device 41 into the nasal cavity. FIG. 24E: illustrates step 5: The nasal applicator device 10, now released from the nasal cavity device 41, tip guide 35 and flex clip 39 is removed from the nasal cavity leaving the nasal cavity device 41 in the olfactory cleft and held in place, or stabilized, by the stabilizer 22 of the top stabilizer assembly comprising the tip guide 35, the flex clip 39, and the stabilizer 22. FIG. 24F: illustrates step 6: The septum clip 43 is applied clamping the nasal cavity device 41 to the nasal septum.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

Every device, system, formulation, combination of components, or method described or exemplified herein can be used to practice the invention, unless otherwise stated.

Whenever a range is given in the specification, for example, a spatial range, a rotation range, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention.

What is claimed is:

1. A nasal applicator device for precision placement of a nasal cavity device in an olfactory cleft of a subject comprising:
a handle extending between a handle distal end and a distal proximal end;
one or more controllers operably connected to the handle;
an applicator arm connected to the handle distal end,
a distal end effector configured to operably connect to the applicator arm to guide placement of the nasal cavity device;
a stabilizer operably connected to an applicator arm distal end, wherein during use the stabilizer is configured to stably immobilize at least a portion of the nasal cavity device within the olfactory cleft of the subject.

2. The nasal applicator device of claim 1, further comprising:
a hollow tip positioned at the applicator arm distal end configured to stably immobilize the nasal cavity device in the applicator arm.

3. The nasal applicator device of claim 1, wherein the distal end effector comprises a tip guide and/or a flex clip, wherein the tip guide and/or the flex clip are operably connected to the applicator arm distal end.

4. The nasal applicator device of claim 1, wherein the applicator arm comprises an applicator channel cover releasably connected to the nasal cavity device.

5. The nasal applicator device of claim 1, wherein the stabilizer is part of a top stabilizer assembly; wherein the top stabilizer assembly comprises the flex clip, the tip guide, and the stabilizer and wherein the flex clip, the tip guide and the stabilizer are operably connected to one another.

6. The nasal applicator device of claim 1, further comprising:
an applicator channel longitudinally aligned with the applicator arm, wherein the applicator channel longitudinally extends from the handle distal end and is configured to connect to a nasal endoscope to visualize the nasal cavity.

7. The nasal applicator device of claim 1, wherein the tip guide has a curved geometry or malleability to accommodate a mechanical rotation of the nasal cavity device at an external angle of between 45° and 90° for controllable placement of at least a portion of the nasal cavity device within the olfactory cleft of the subject.

8. The nasal applicator device of claim 1, wherein at least one of the one or more controllers are configured to advance and/or retract the nasal cavity device within the nasal cavity, engage the stabilizer, release the top stabilizer assembly, retract the flex clip, secure the flex tip to the tip guide, rotate the applicator channel cover or any combination thereof.

9. The nasal applicator device of claim 8, wherein at least one of the one or more controllers are configured to advance and/or retract the nasal cavity device between 10 mm to 40 mm so as to place at least a portion of the nasal cavity device within the olfactory cleft of the subject.

10. The nasal applicator device of claim 1, wherein the one or more controllers is selected from the group consisting of: a thumb screw, a finger slide, an advancement wheel, and a deployment trigger for nasal cavity device detachment.

11. The nasal applicator device of claim 10, wherein the finger slide, the advancement wheel, or the deployment trigger is configured to detach the nasal cavity device, the top stabilizer assembly, the stabilizer, or any combination thereof from the nasal applicator device such that the nasal applicator device can be removed from the nasal cavity and the nasal cavity device, the top stabilizer assembly, the stabilizer, or any combination thereof remains in the nasal cavity of the subject.

12. The nasal applicator device of claim 1, wherein the stabilizer, the distal end effector, or the top stabilizer assembly is configured to be deployed at the opening of the olfactory cleft of the subject.

13. The nasal applicator device of claim 1, wherein the stabilizer comprises a dried compressed sponge, an inflatable balloon, or any combination thereof.

14. The nasal applicator device of claims 13, wherein the dried compressed sponge has an inflated volume with contact with a mucosal fluid.

15. The nasal applicator device of claim 13, wherein the inflatable balloon is in operable connection with a controller to provide a mechanical inflation having an inflated diameter of up to 6 mm.

16. The nasal applicator device of claim 1, wherein the nasal cavity device comprises an electrophysiological device having one or more electrodes at least partially disposed on a distal tip of the nasal cavity device.

17. The nasal applicator device of claim 1, wherein the applicator arm distal end is configured to operably connect to the nasal cavity device.

18. A method of electrophysiologically interfacing a nasal cavity device with a biological tissue of a subject, the method comprising the steps of:
providing the nasal applicator device of claim 1;
operably connecting a nasal cavity device with the applicator arm distal end to provide a loaded nasal applicator device;
hand-positioning the handle to introduce the loaded nasal applicator device to a nasal cavity, including an anterior opening of the cribriform plate;
first engaging the one or more controllers to advance the nasal cavity device between 10 mm to 40 mm so that at least a portion of the nasal cavity device is positioned within an olfactory cleft of the subject;
deploying the stabilizer at the opening of an olfactory cleft of the subject so that at least a portion of a distal end of the nasal cavity device is in electrical contact with the sphenoid sinus wall;
second engaging the one or more controllers to release the nasal cavity device and the stabilizer from the nasal applicator device; and
removing the nasal applicator device from the nasal cavity,
thereby electrophysiologically interfacing the nasal cavity device with the biological tissue.

19. The method of claim 18, further comprising:
holding a nasal endoscope with a second hand to visualize a position of the nasal applicator device in the nasal cavity.

20. The method of claim 19, further comprising the step of:
simultaneously inserting the nasal applicator device and the nasal endoscope into the subject's nasal cavity.

21. The method of claim 18, wherein the step of second engaging the one or more controllers further comprises:
retracting the top stabilizer assembly from the applicator arm; and
rotating an applicator channel cover of the applicator arm;
thereby releasing the top stabilizer assembly and the nasal cavity device from the nasal applicator device.
